(19) United States
(12) Patent Application Publication
(10) Pub. No.: US 2017/0314007 A1
(43) Pub. Date: Nov. 2, 2017

(54) MEDICAMENT DESIGN POCKET OF ORNITHINE DECARBOXYLASE AND APPLICATION OF MEDICAMENT DESIGN POCKET

(71) Applicant: CHINA THREE GORGES UNIVERSITY, Yichang, Hubei (CN)

(72) Inventor: Sen LIU, Yichang, Hubei (US)

(73) Assignee: CHINA THREE GORGES UNIVERSITY, Yichang, Hubei (CN)

(21) Appl. No.: 15/309,801

(22) PCT Filed: Sep. 29, 2015
(86) PCT No.: PCT/CN2015/091044

§ 371 Date: Nov. 9, 2016

(30) Foreign Application Priority Data
Sep. 30, 2014 (CN) 201410522192.2
Sep. 30, 2014 (CN) 201410524065.6
Sep. 30, 2014 (CN) 201410524151.7
Sep. 30, 2014 (CN) 201410525191.3
Sep. 30, 2014 (CN) 201410525202.8

(51) Int. Cl. C12N 9/88; C12Q 1/527; C07D 213/53; C07D 251/54; C07D 239/70; G06F 19/16; C07C 233/81

(52) U.S. Cl. CPC C12N 9/88; G06F 19/16; C12Q 1/527; C12Y 401/01017; C07C 233/81; C07D 251/54; C07D 239/70; C07D 213/53; G01N 2500/00

(57) ABSTRACT

The present invention relates to a medicament design pocket of ODC. Based on the crystal structure of human ODC, the binding site area of putrescine and PLP ligand on the ODC homodimer interface is the medicament pocket, which is used for screening or designing or modifying inhibitors of human ODC, or screening or designing or modifying inhibitors of non-human ODC, or screening or designing or modifying protein inhibitor highly homologous to the binding site of putrescine and pyridoxal phosphate on the interface of ODC homodimer. The invention also provides the structure of the inhibitor and its application thereof. The technical solutions in the invention provide reliable theoretical basis for the research and development of human ODC, the prevention, treatment and diagnosis of tumors and pathogenic microbial infections, and the research and development and preparation of medicaments for the treatment of tumors or pathogenic microbial infections.

### FIELD

The present invention belongs to the field of biomedicine and relates to a medicament design pocket of ODC (ornithine decarboxylase). The medicament design pocket is used in discovering ODC inhibitors; moreover, this invention also provides a method for inhibiting ODC by small molecule inhibitors.

### BACKGROUND

Protein is one of the main components of organisms and the main substance for completing a variety of life activities. Among all proteins, proteases are essential for the life activities, and almost ail in vivo biochemical reaction processes of organisms need the catalyzation of proteases. The in vivo activities of proteases have strict regulation mechanism; and once the mechanism dysfunctions, excessively high or low activity or inactivation of proteases may occur, which will lead to various diseases. Therefore, it is of great theoretical and practical significance to regulate the activity of protease by medicaments to restore and maintain the normal levels. The structure-based medicament design is a very important method for designing protein-targeted medicaments.

Polyamines are a kind of positively charged cationic small molecules generating from amino acid metabolism, which exist in all organisms and play essential roles for the cell growth, differentiation, survival and -normal biological functions. Polyamines are positively charged so that they can regulate a wide range of biological processes by forming electrostatic interactions with negatively charged biomacromolecules (DNA, RNA, proteins, cell membranes, etc.), including chromosome structure formation, DNA synthesis and stability, DNA replication, transcription and translation, protein phosphorylation, ribosome formation, ion channel and membrane surface receptor regulation, free radical scavenging, etc. There are many kinds of natural polyamines. In mammals, there are three kinds of natural polyamines; putrescine, spermidine and spermine, which are essential for normal mammalian growth and development Since polyamines have important biological functions, their intracellular levels are strictly regulated. In rapidly proliferating cells (such as tumor cells), the level of polyamides and ODC expression level will increase and become unregulated. With the increased polyamine level, the cell proliferation will accelerate, the apoptosis will decrease and the expression levels of genes related to tumor invasion and metastasis will increase. Therefore, the regulation of polyamines has become an important method for tumor therapy and medicament research and development.

The starting substrate for polyamine metabolism is ornithine, which is the reaction product of arginine catalyzed by arginase in the urea cycle. ODC is the first enzyme in the polyamine synthesis pathway, which catalyzes the reaction from ornithine to putrescine, and this catalytic reaction is also a rate-limiting step in the polyamine synthesis pathway. Therefore, the synthesis of ODC inhibitor and inhibition of putrescine generation is a tumor treatment approach that is worthy of attentions. Besides, since the pathogenic microorganisms require normal levels of polyamines, ODC inhibitor has become an important target for pathogenic microorganisms (such as Trypanosoma brucei that causes African trypanosomiasis).

Currently, DFMO (a-difluoromethyl ornithine), the ODC's inhibitor, has been used clinically for adjuvant chemotherapy of cancer. But it has a weak ability to hind ODC with high concentration, and it covalently bonds with ODC to form a suicide inhibitor, with very strong toxic and side effects. Therefore, it is urgent to develop a new ODC inhibitor with better effect.

### SUMMARY

One object of the invention is to identify a new medicament design pocket for ODC protease, which is used for screening and designing of novel ODC inhibitors.

0The technical solution of the invention is to find out and determine the binding pocket used for medicament design with protein medicament design pocket analysts software by analyzing the crystal structure of ODC, and implement medicament screening and validation for she binding pocket.

According to the above solution, the substrate and PLP ligand-binding pocket area are analyzed on the basis of the crystal structure of human ODC. With the Pocket software, the region of binding site between putrescine substrate and PLP ligands on the ODC homodimer interface is identified as the medicament design pocket.

The schematic structure of the homodimer is shown in FIG. 1, of which, one chain is displayed as the surface, and the other chain is displayed as cartoon.

The binding site of ODC substrate (putrescine) and PLP is shown in FIG. 2, of which, one chain is displayed as the surface, and the other chain is displayed as cartoon, and the putrescine and PLP are in rod shapes.

The schematic diagram of this medicament design pocket is shown in FIG. 3. The pocket is shown as a translucent surface with surrounding stick-like amino acid residue side chains on ODC homodimer. The residues constituting this pocket include Phe65, Ala67, Lys69, Cys70, Asp88, Ala90, Ala 111, Asn112, Pro113, Thr132, Arg154, Cys164, Arg165, Leu166, Phe170, Phe196, His97, Gly199, Ser200, Gly201, Gly235, Gly236, Glv237, Phe238, Pro239, Glu274, Pro275, Gly276, Arg277, Tyr278, Asn327, Cys328, Tyr133, Asp332, His333, Ala388, Tyr389 on one protein monomer and Tyr323, Thr359, Cys360, Asp361, Gly362, Leu363, Phe397, Asn398 on another protein monomer. The sticks in the pocket are the PLP, putrescine and the known inhibitor DFMO of ODC.

The applicable object of the invention is human ODC, however, since ODC from different sources has homology, the inhibitor that can act on the medicament pocket in the invention can also act on other non-human ODC and proteins highly homologous to ODC substrate (Putrescine) and PLP binding pocket.

Another object of the invention is to provide a novel inhibitor against ODC by computer-assisted high throughput screening of medicaments, which is used to ODC inhibitors for preparing medicaments for the treatment of tumors and pathogenic microbial infections. Specifically, the structural formula of the inhibitor is;

Wherein, R₁, R₂ are

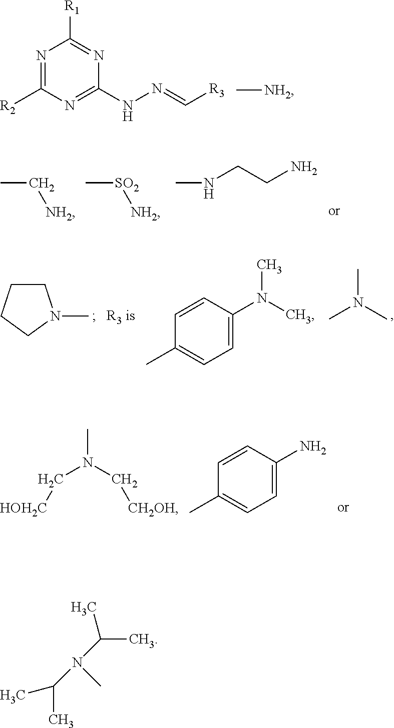

Further, preferably the structural formula of the inhibitor is

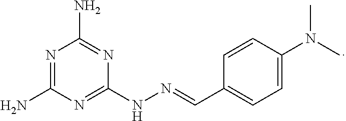 [SMILES: CN(C)c1ccc(/C=N/Nc2nc(N)nc(N)n2)cc1]

The structural formula also can be;

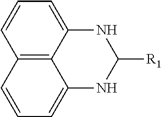

### Wherein, R₁ is

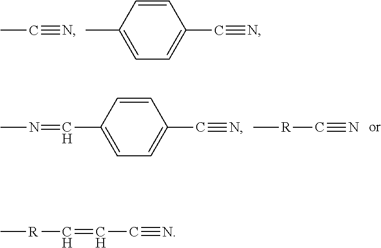

Further, preferably the structural formula of the inhibitor is

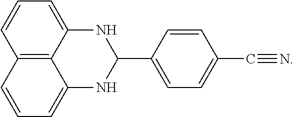 [SMILES: N#Cc1ccc(C2Nc3cccc4cccc(c34)N2)cc1]

The structural formula also can be;

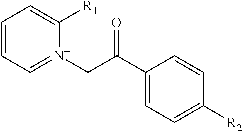

wherein, R₁ is in ortho or meta or para position; R₂ is in para or meta position.
Wherein, R₁ includes

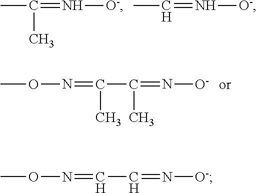

R₂ includes

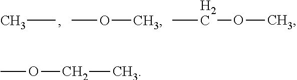

Further, preferably the structural formula is:

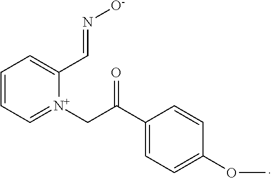 [SMILES: COc1ccc(C(=O)C[n+]2ccccc2/C=N/[O-])cc1]

The above structural formula also can be:

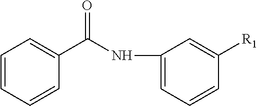

Wherein, R₁ is in ortho or meta or para position, R₁ alkyl esters, alkyl ethers, alkyl aldehydes or alkyl ketones.
Wherein, R₁ includes

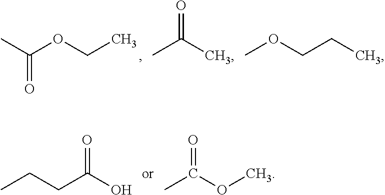

Further, preferably the structural formula is:

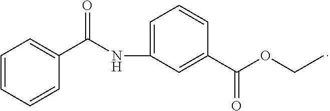 [SMILES: CCOC(=O)c1cccc(NC(=O)c2ccccc2)c1]

In the present invention, the above inhibitor is used to inhibit ODC, of which, the ODCs are a human ODC, a non-human ODC or a protein highly homologous to the putrescine substrate and PLP binding sites of human ODC.

The method for applying the above inhibitor to inhibit ODC, comprising the following steps;

1) The Construction of ODC Prokaryotic Expression Plasmid

The ODC gene sequence is inserted into pET28a plasmid by BamH I and Xho I cleavage sites to construct pET28a-hODC plasmid, which is verified by DNA sequencing;

2) ODC Protein Expression

The plasmid pET28a-hODC constructed in the step 1) is transformed into Escherichia coli BL21 strain by CaCl2 method and screened by kanamycin, and then the strains grown on the kanamycin-containing Luria-Bertani (LB) culture plate are inoculated to kanamycin-containing LB liquid medium, cultured to logarithmic phase at 37° C and 250 rpm, and then IPTG is added to 0.5 mM for induced expression 4 hours at 28° C., finally, centrifuged to collect bacteria;

3) Purification of ODC Protein

The bacteria collected in step 2) are re-suspended with lysate solution, then cells are lysed by ultrasonic method. After lysis bufferis centrifuged at 12000 rpm/min at 4° C., the supernatant is retained; finally the supernatant is bound and purified using Ni-NTA His labeled protein binding packing, to get human ODC protein. The ODC protein elution buffer is 50 mM Tris/HCl, pH 8.0, 300 mM NaCl, 1 mM DTT, 100 mM imidazole;

4) Detection of ODC Protein Activity

400 uL substrate reaction mixture and 50 ug ODC protein are added to EP tube, mixed evenly, and the EP tube is placed in 37° C. water bath for 30 min; 400 uL 10% TDA is added to terminate the reaction, centrifuged 5 min at 5000 rpm at room temperature, then 100 uL supernatant is fetched and mixed with 200 uL of 4 mol/L NaOH, 400 uL of n-amyl alcohol is added to mix well, centrifuged 5min at 2000 rpm, then 200 uL of the supernatant is transferred to a new EP tube, and 200 uL of 0.1 mol/L sodium tetraborate (pH 8.0) is added to mix evenly, and 200 uL of 10 mmol/L trinitrobenzene sulfonic acid is added to mix fully, and then 400 uL DMSO is added to fix fully for 1 min, centrifuged 5 min at 3000 rpm; finally the supernatant is fetched to 96-well plate and its absorbance at 426 nm is detected by a microplate reader, to get the OD value with out adding enzyme;

5) Detection of Inhibitory Activity of Inhibitor for ODC Protein

According to the method described in step 4), after adding 400 μL substrate reaction mixture, the ODC inhibitor is added immediately, and subsequent procedures are the same as the step 4);

The ODC inhibition ratio is calculated according to the following formula;

Control difference=the mean OD value of the control group adding the inhibitor−the mean OD value of the control group without adding inhibitor, of which, the inhibitor added in step 5) in the control group is DFMO inhibitor;

Experimental difference= the mean OD value of the experiment group adding the inhibitor−the mean OD value of the experiment group without adding inhibitor.

ODC inhibition ratio=[(control difference-experimental difference)/control difference]×100%.

The lysis buffer described in step 3) is a mixture of 50 mM Tris/HCl, pH 8.0, 300 mM NaCl, 1 mM DTT, 1 mM PMSF and 5 mM imidazole.

The substrate reaction mixture in step 4): dissolve 17.57 ul β-mercaptoethanol, 55.84mg of 1.5 mM EDTA disodium salt, 75 nM PLP stock solution and 2 mM ornithine hydrochloride in 150 mM PBS (pH 7.1).

According to the above technical solution, the putrescine substrate and PLP ligand are analyzed based on human ODC crystal structure using Pocket protein medicament pocket analysis software, to establish a theoretical model of the protein pocket, then 190,000 small molecules in the SPECS medicament library are docked into the protein pocket model using the protein docking program DOCK, to screen out small molecules containing at least 15 non-hydrogen heavy-atoms, at least 2 hydrogen bonds, at least one hydrophobic center and docking score no more than −10; and then the above small molecules are docked to the above protein pocket for computation using the protein-small molecule docking program Autodock, and finally small molecules with docking score below −7 are selected.

The present invention also presides a composition comprising an inhibitor or an analog thereof and a pharmaceutical acceptable carries with the effective amount for inhibiting ODC. Preferably the composition is a pharmaceutical composition comprising an inhibitor or analogue thereof and a pharmaceutically acceptable carrier with the effective amount for treatment. More preferably, the pharmaceutical composition is the one that can treat or prevent the disease that is responsive to ODC inhibition, wherein the ODC is preferably a human ODC; it further comprises an inhibitor or analogue thereof and a pharmaceutical acceptable carrier with the effective amount for preventing and treating the diseases that produce response to ODC inhibition provided in the invention,

The inhibitors or analogs thereof and the aforesaid compositions in the invention can be used to inhibit ODC, preferably human-derived ODC, and the inhibition is for therapeutic or non-therapeutic purposes. Preferably the inhibitor or analogs thereof in the invention are used to prepare medicaments for Inhibiting ODC, preferably human-derived ODC. Therefore, the present invention also provides a method of inhibiting ODC activity, comprising the use of effective amount of inhibitor or analogs thereof or above composition, wherein the inhibition is for therapeutic or non-therapeutic purposes.

The present invention also provides a method of treating a disease responsive to ODC inhibition, comprising the use of effective amount of inhibitor or analogs thereof or above composition that can inhibit ODC (preferably human-derived ODC) for individuals that need treatment or prevention.

The inhibitors or analogs thereof in the invention can be used for producing pharmaceuticals or pharmaceutical compositions that can treat diseases responsive to ODC inhibition, and ODC or analogues thereof can inhibit the activity of ODC. The diseases Include tumors or pathogenic microbial infections, preferably the aforesaid tumors. Protozoal infection refers to a tumor or a pathogenic microbial infection disease that is responsive to inhibition of ODC.

In the invention, the aforesaid inhibitor is used to prepare medicaments for treatment of tumors.

Further, the aforesaid inhibitor is used to prepare medicaments for treatment of pathogenic microbial infections.

### BRIEF DESCRIPTION OF THE DRAWINGS

FIG. 1 shows a schematic diagram of ODC homodimer.

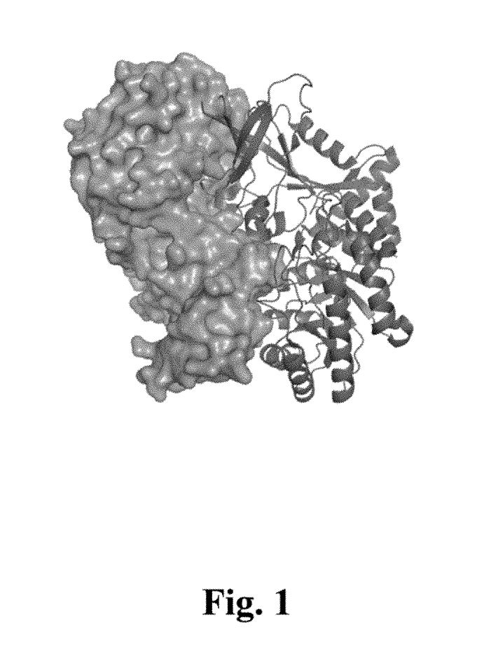

FIG. 2 shows a schematic diagram of the binding site of ODC substrate putrescine and PLP.

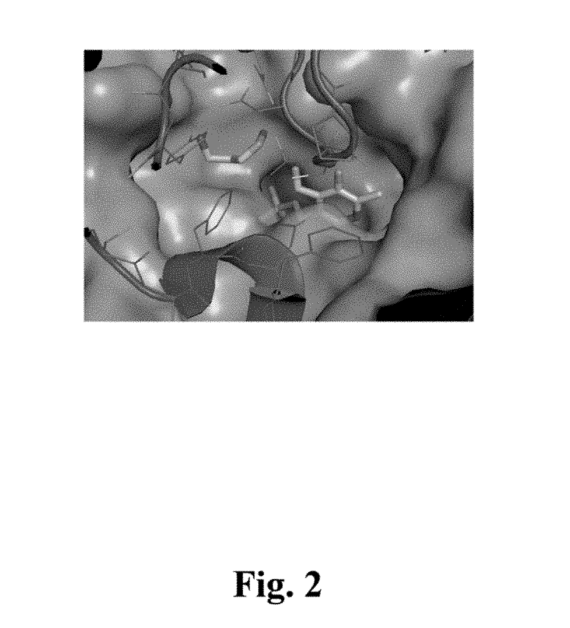

FIG. 3 shows a schematic diagram of a medicament design pocket in the invention.

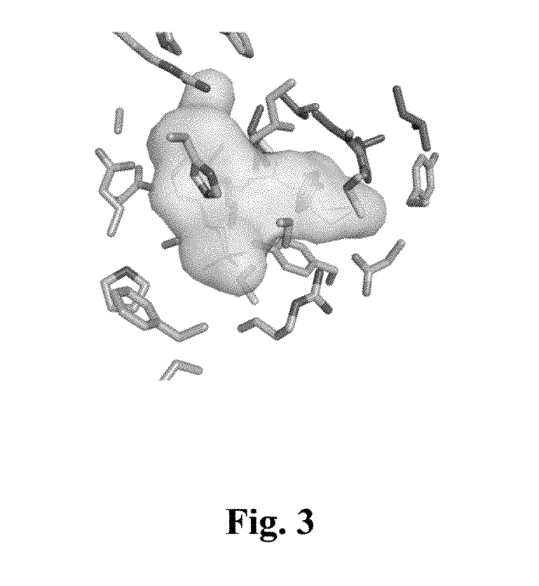

FIG. 4 shows a histogram of inhibitory activity of α-difluoromethylornithine (DFMO) inhibitor on human ODC.

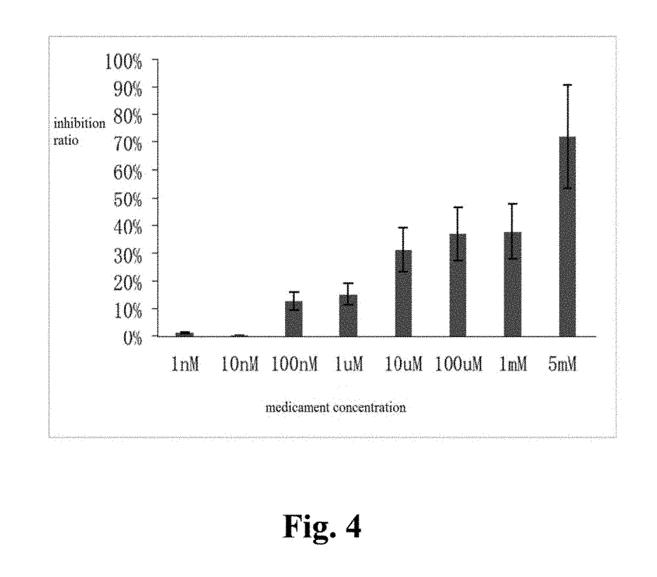

FIG. 5 shows a histogram of inhibitory activity of 4-(2-3-dihydro-1H-pyrimidin-2-yl) benzonitrile inhibitor on human ODC.

FIG. 6 shows a model of binding of 4-(2-3-dihydro-1H-pyrimidin-2-yl) benzonitrile inhibitor and ODC.

FIG. 7 shows a histogram of inhibitory activity of ethyl-3-(benzoylamino) methyl benzoate inhibitor on human ODC.

FIG. 8 shows a model of binding of ethyl-3-(benzoylamino) methyl benzoate inhibitor and ODC.

FIG. 9 shows a histogram of inhibitory activity of 4-(dimethylamino)-benzaldehyde-(4,6-diamino-1,3,5) triazine inhibitor on human ODC.

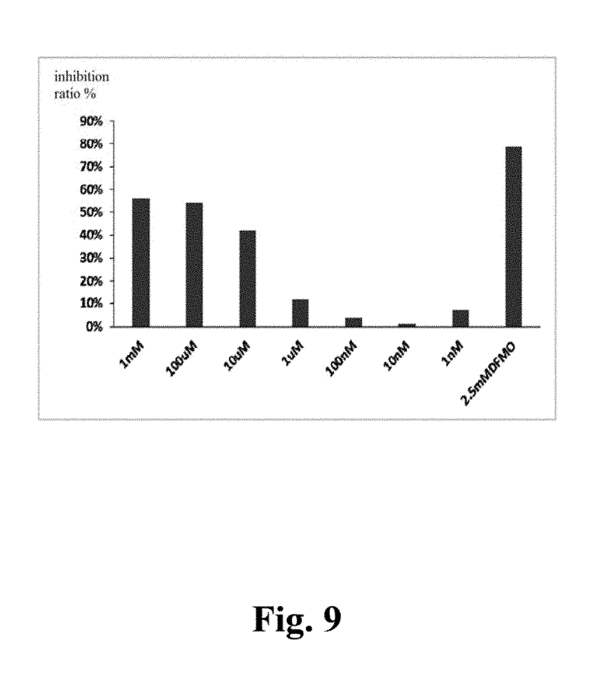

FIG. 10 shows a model of binding of 4-(dimethylamino)-benzaldehyde-(4,6-diamino-1,3,5) triazine inhibitor and ODC.

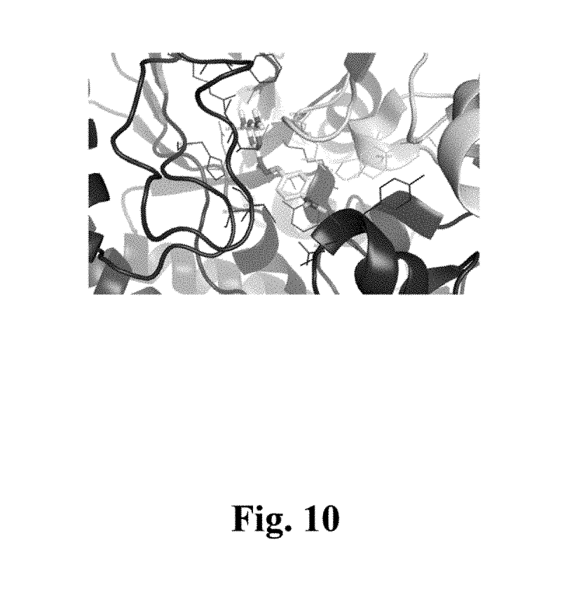

FIG. 11 shows a histogram of inhibitory activity of 2-[(hydroxyimino) methyl]-1-[2-(4-methoxyphenyl) -2-oxoethyl] pyridinium inhibitor on human ODC.

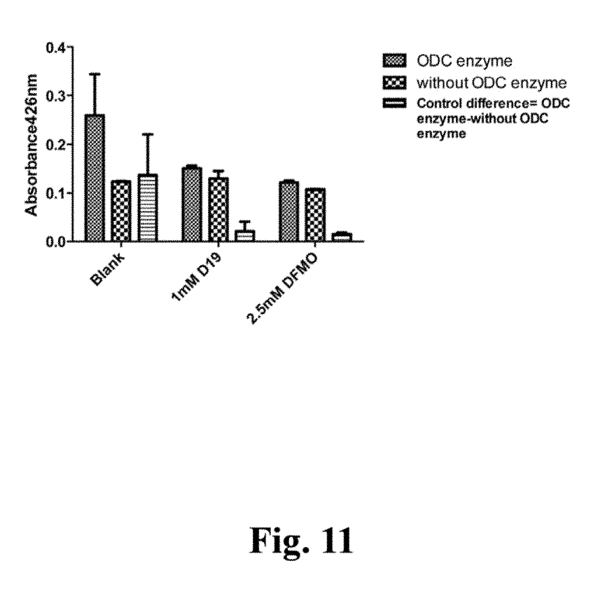

FIG. 12 shows a model of binding of 2-[(hydroxyimino) methyl]-1-[2-(4-methoxyphenyl)-2-oxoethyl] pyridinium inhibitor and ODC.

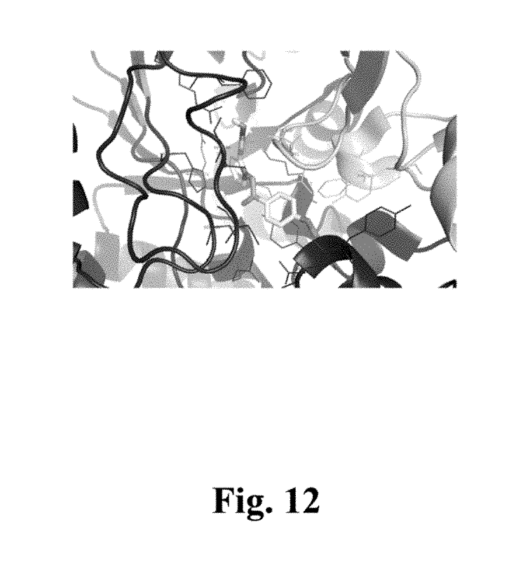

### DETAILED DESCRIPTION

### Embodiment 1

In the present invention, the substrate and PLP ligand are analyzed based on human ODC crystal structure using Pocket protein medicament pocket analysis software, to establish a theoretical model of the protein pocket, then 190,000 small molecules in the SPECS medicament library are docked into the protein pocket model using the protein docking program DOCK, to screen out small molecules containing at least 15 non-hydrogen heavy-atoms, at least 2 hydrogen bonds, at least one hydrophobic center and docking score no more than -10; and then the above small molecules are docked to the above protein pocket for computation using the protein-small molecule docking program Autodock, and finally small molecules with docking score below -7are selected.

In the experimental validation, the inhibitor involved is 4-(2-3-dihydro-1H-pyrimidin-2-yl) benzonitrile.

In the present invention, the ODC inhibitor having a similar inhibitory effect that is obtained by side chain addition, deletion and fragment replenishment using 4-(2-3-dihydro-1H-pyrimidin-2-yl) benzonitrile as a parent body is not excluded.

In the following, the experimental procedure of inhibition is described by taking 4-(2-3-dihydro-1H-pyrimidin-2-yl) benzonitrile as an example.

The structure of 4-(2-3-dihydro-1H-pyrimidin-2-yl) benzonitrile:

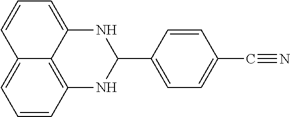 [SMILES: N#Cc1ccc(C2Nc3cccc4cccc(c34)N2)cc1]

1. Construction of Prokaryotic Expression Plasmid of Human ODC.

The gene sequence of human ODC is inserted in to pET28a plasmid by BamH I and Xho I cleavage sites, to construct pET28a-hODC plasmid, which is verified by DNA sequencing. The gene sequences of human ODC:

atgaacaactttggtaatgaagagtttgactgccacttcctcgatgaagg
ttttactgccaaggacattctggaccagaaaattaatgaagtttcttctt
ctgatgataaggatgccttctatgtggcagacctgggagacattctaaag
aaacatctgaggtggttaaaagctctccctcgtgtcacccccttttatgc
agtcaaatgtaatgatagcaaagccatcgtgaagacccttgctgctaccg
ggacaggatttgactgtgctagcaagactgaaatacagttggtgcagagt
ctgggggtgcctccagagaggattatctatgcaaatccttgtaaacaagt
atctcaaattaagtatgctgctaataatggagtccagatgatgacttttg
atagtgaagttgagttgatgaaagttgccagagcacatcccaaagcaaag
ttggttttgcggattgccactgatgattccaaagcagtctgtcgtctcag
tgtgaaattcggtgccacgctcagaaccagcaggctccttttggaacggg
cgaaagagctaaatatcgatgttgttggtgtcagcttccatgtaggaagc
ggctgtaccgatcctgagaccttcgtgcaggcaatctctgatgcccgctg
tgtttttgacatgggggctgaggttggtttcagcatgtatctgcttgata
ttggcggtggctttcctggatctgaggatgtgaaacttaaatttgaagag
atcaccggcgtaatcaacccagcgttggacaaatactttccgtcagactc
tggagtgagaatcatagctgagcccggcagatactatgttgcatcagctt
tcacgcttgcagttaatatcattgccaagaaaattgtattaaaggaacag
acgggctctgatgacgaagatgagtcgagtgagcagacctttatgtatta
tgtgaatgatggcgtctatggatcatttaattgcatactctatgaccacg
cacatgtaaagccccttctgcaaaagagacctaaaccagatgagaagtat
tattcatccagcatatggggaccaacatgtgatggcctcgatcggattgt
tgagcgctgtgacctgcctgaaatgcatgtgggtgattggatgctctttg
aaaacatgggcgcttacactgttgctgctgcctctacgttcaatggcttc
cagaggccgacgatctactatgtgatgtcagggcctgcgtggcaactcat
gcagcaattccagaaccccgacttcccacccgaagtagaggaacaggatg
ccagcaccctgcctgtgtcttgtgcctgggagagtgggatgaaacgccac
agagcagcctgtgcttcggctagtattaatgtgtag.←

The aforesaid human ODC sequences used have a change of base compared with the sequences in the database (http://www.ncbi.nlm.nih.gov/nuccore/NM_002539.1)(The bold marked C is T in the database sequence, and the corresponding amino acid is changed from arginine to cysteine), but its activity is not affected.

2. Expression of Human ODC Protein

The plasmid pET28a-hODC constructed is transformed into Escherichia coli BL21strain by CaCl₂ method and screened by kanamycin, and then the strains grown on the kanamycin-containing Luria-Bertani (LB) culture plate are inoculated to kanamycin-containing LB liquid medium, cultured to logarithmic phase at. 37° C and 250 rpm, and then IPTG is added to 0.5 mM for induced expression 4 hours at 28° C., finally, centrifuged to collect bacteria;

3. Purification of Human ODC Protein

The bacteria collected in the above step are re-suspended with lysis buffer (50 mM Tris/HCl, pH 8.0, 300 mM NaCl, 1 mM DTT, 1 mM PMSF, 5 mM imidazole), then cells are lysed by ultrasonic method. After lysis buffer is centrifuged at 12000 rpm/min at 4° C., the supernatant is retained; finally, the supernatant is bound and purified using Ni-NTA His labeled protein binding packing., to get human ODC protein. The ODC protein elation butter is 50 mM Tris/HCl, pH 8.0, 300 mM NaCl, 1 mM DTT 100 mM imidazole.

4. Detection of ODC Protein Activity

400 uL substrate reaction mixture (17.57 ul of β-mercaptoethanol, 55.84 mg of 1.5 mM EDTA disodium salt, 75 nM PLP stock solution, 2 mM ornithine hydrochloride are dissolved in 150 mM PBS (pH 7.1)) and 50 ug ODC protein are added to a 1.5 mL of EP tube, mixed evenly, and the EP tube is placed in 37° C water bath for 30 min; 400 uL 10% TDA is added to terminate the reaction, centrifuged 5 min at 5000 rpm at room temperature, then 100 uL supernatant is fetched and mixed with 200 uL of 4 mol/L NaOH, 400 uL of n-amyl alcohol is added to mix well, centrifuged 5 min at 2000 rpm then 200 uL of the supernatant is transferred to a new EP tube, and 200 uL of sodium tetraborate (0.1 mol/L, pH 8.0) is added to mix evenly, and 200 uL of 10 mmol/L trinitrobenzene sulfonic acid is added to mix fully, and then 400 uL DMSO is added to fix fully for 1 min, centrifuged 5 min at 3000 rpm, finally the supernatant is fetched to 96-well plate and its absorbance at 426 nm is detected by a microplate reader.

5. Detection of Inhibitory Activity of 4-(2-3-dihydro-1H-pyrimidin-2-yl) Benzonitrile Inhibitor for Human ODC Protein.

According to the above detection steps, after adding 400 μL substrate reaction mixture, small molecule medicament is added and mixed immediately, aid subsequent procedures are the same.

The ODC inhibition ratio is calculated according to the following formula;

Control difference=the mean OD value of the control group adding the inhibitor−the mean OD value of the control group without adding inhibitor

Experimental difference=the mean OD value of the experiment group adding the inhibitor−the mean OD value of the experiment group without adding inhibitor

ODC inhibition ratio=[(control difference−experimental difference)/control difference]×100%.

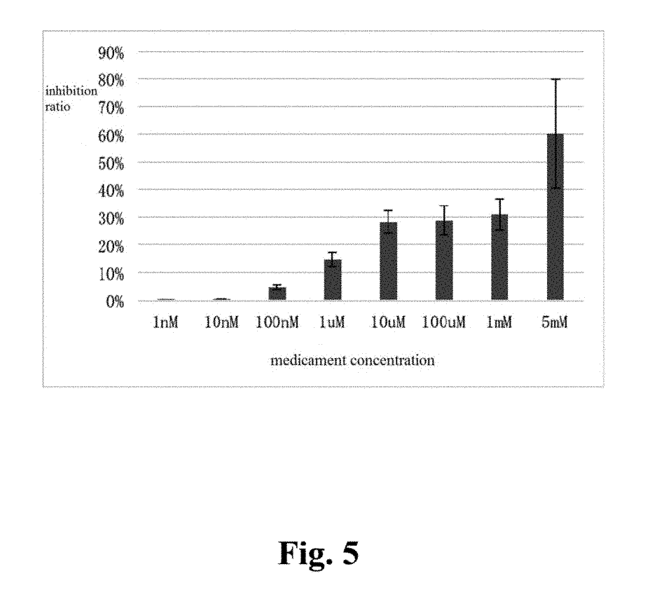

FIG. 5 shows a histogram of inhibitory activity of 4-(2-3-dihydro-1H-pyrimidin-2-yl) benzonitrile inhibitor on human ODC. The results show that, this inhibitor has the ability to inhibit human ODC activity within the range of 1 nM-5 mM, and the higher the concentration, the stronger tire inhibitory ability.

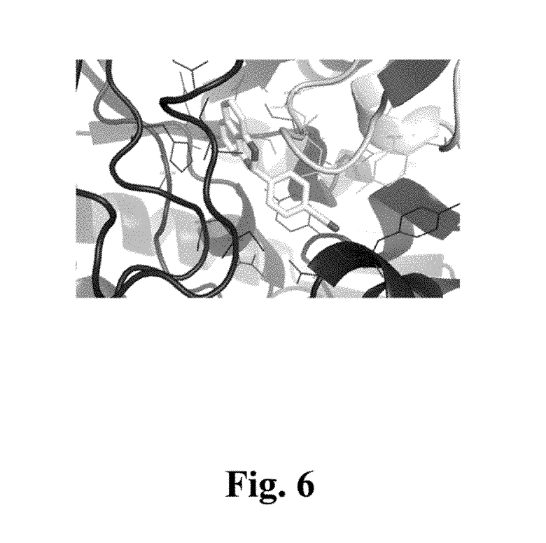

FIG. 6 shows a binding model of 4-(2-3-dihydro-1H-pyrimidin-2-yl) benzonitrile inhibitor with ODC. As shown from the FIG., the black represents a chain of ODC homodimer, and the grey represents another chain. The small molecule medicament is shown as a rod-like model that binds to the dimer interface pocket. The residues that may be involved in the interaction within 4 angstroms around the small molecule medicament are marked to show the side chains.

### Embodiment 2

The small molecule inhibitor involved, in the invention is ethyl-3-(benzoylamino) methyl benzoate inhibitor, having the structural formula as follows;

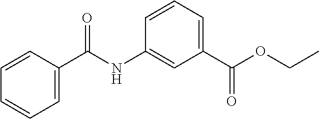 [SMILES: CCOC(=O)c1cccc(NC(=O)c2ccccc2)c1]

In the embodiment the ODC inhibitor having a similar inhibitory effect that is obtained by side chain addition, deletion and fragment replenishment using this medicament as a parent body is not excluded.

The specific steps are the same as embodiment 1.

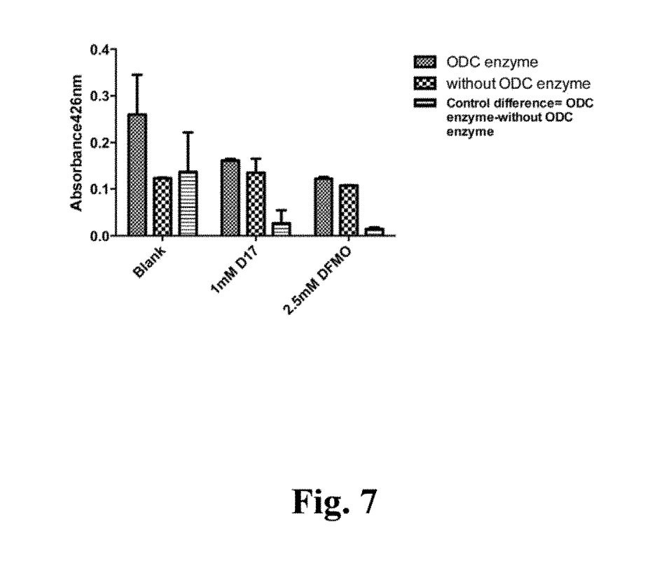

FIG. 7 shows a histogram of inhibitory activity of ethyl-3-(benzoylamino) methyl benzoate inhibitor on human ODC. By comparing the histogram of inhibitory activity of ethyl-3-(benzoylamino) methyl benzoate inhibitor with the DFMO inhibitor on human ODC, results show that the inhibitory capacity of 1 mM of this medicament (No. D17) on human ODC is equivalent to that of 2.5 mM DFMO.

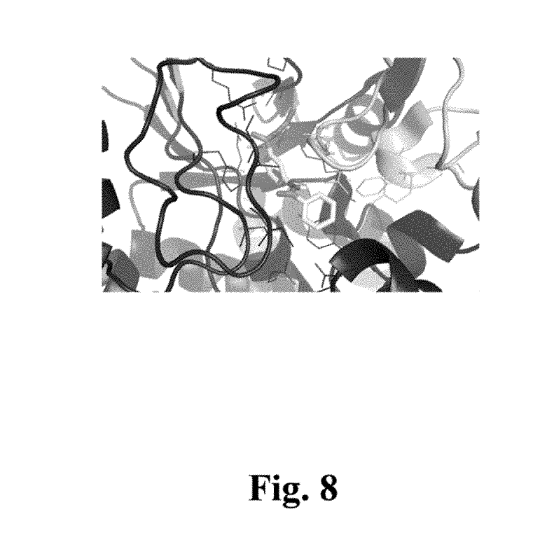

FIG. 8 shows a binding model of ethyl-3-(benzamido) methyl benzoate inhibitor and ODC. As shown from the figure, the black represents a chain of ODC homodimer, and the grey represents another chain. The small molecule medicament is shown as a rod-like model that binds to the dimer interface pocket. The residues that may be involved in the interaction within 4 angstroms around the small molecule medicament are marked to show the side chains.

### Embodiment 3

The small molecule inhibitor involved in the invention is 4-(dimethylamino)-benzaldehyde-(4,6-diamino-1,3,5) triazine inhibitor, having the structural formula as follows:

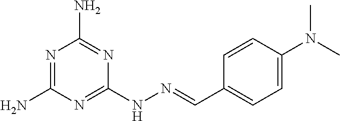 [SMILES: CN(C)c1ccc(/C=N/Nc2nc(N)nc(N)n2)cc1]

In the embodiment, the ODC inhibitor having a similar inhibitory effect that is obtained by side chain addition, deletion and fragment replenishment using 4-(dimethylamino)-benzaldehyde-(4,6-diamino-1,3,5) triazine as a parent body is not excluded.

The specific steps are the same as embodiment 1.

By comparing the histogram of inhibitory activity of 4-(dimethylamino)-benzaldehyde-(4,6-diamino-1,3,5) triazine inhibitor with tire DFMO inhibitor on human ODC, results show that this medicament has inhibitory effect on human ODC activity within the concentration range of 1 nM-1 mM.

The results of binding model of 4-(dimethylamino)-benzaldehyde-(4,6-diamino-1,3,5) triazine inhibitor with ODC show that, the small molecule medicament is in a rod-like model. The two monomers of homodimer are displayed as black and light gray cartoon models respectively. The residues that may be involved in the interaction within 4 angstroms around the small molecule medicament are marked to show the side chains.

### Embodiment 4

The small molecule inhibitor involved in the invention is 2-[(hydroxyimino)methyl]-1-[2-(4-methoxyphenyl)-2-oxoethyl] pyridinium inhibitor, having the structural formula as follows:

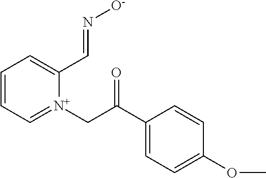 [SMILES: COc1ccc(C(=O)C[n+]2ccccc2/C=N/[O-])cc1]

In the embodiment, the ODC inhibitor having a similar inhibitory effect that is obtained by side chain addition, deletion and fragment replenishment using 2-[(hydroxyimino) methyl]-1-[2-(4-methoxyphenyl)-2-oxoethyl] pyridinium as a parent body is not excluded.

The specific steps ate the same as embodiment 1.

By comparing the histogram of inhibitory activity of 2-[(hydroxyimino) methyl]-1-[2-(4-methoxyphenyl)-2-oxoethyl] pyridinium inhibitor with the DFMO inhibitor on human ODC, results show that the inhibitory capacity of 1 mM of this medicament (No.D19) on human ODC is equivalent to that of 2.5 mM DFMO.

The results of binding model of 2-[(hydroxyimino) methyl]-1-[2-(4-methoxyphenyl)-2-oxoethyl] pyridinium inhibitor with ODC show that, the black represents a chain of ODC homodimer and the grey represents another chain; the small molecule medicament is in a rod-like model that binds to the dimer interface pocket. The residues that may be involved in the interaction within 4 angstroms around the small molecule medicament are marked to show the side chains.

1. A medicament design pocket of ornithine decarboxylase, wherein medicament molecules that inhibit the ornithine decarboxylase (ODC) activity are screened and designed using a binding site area of putrescine and PLP ligand on an ODC homodimer interface as a medicament pocket based on a crystal structure of human ODC, and after binding to the pocket, the medicament molecule may inhibit the formation of ODC dimer or form an inactive ODC dimer.

2. The application of the medicament design pocket of ODC according to claim 1 in screening or designing or modifying inhibitors of human ODC.

3. The application of the medicament design pocket of ODC according to claim 1 in screening or designing or modifying inhibitors of non-human ODC.

4. The application of medicament design pocket of the ODC according to claim 1 in screening or designing or modifying inhibitors highly homologous to the binding site of putrescine and pyridoxal phosphate on the ODC homodimer interface.

5. (canceled)

6. The inhibitor according to claim 1, wherein the medicament molecule is:

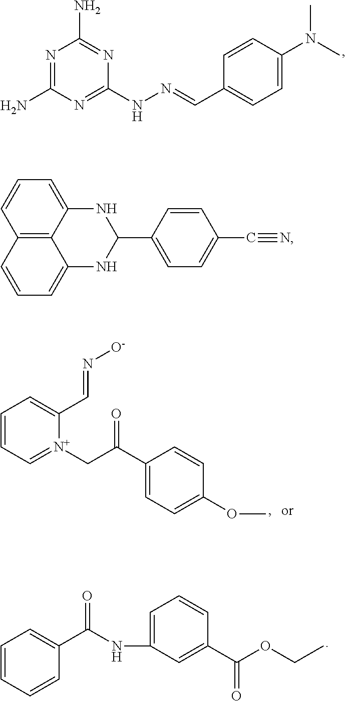 [SMILES: CN(C)c1ccc(/C=N/Nc2nc(N)nc(N)n2)cc1 ; N#Cc1ccc(C2Nc3cccc4cccc(c34)N2)cc1 ; COc1ccc(C(=O)C[n+]2ccccc2/C=N/[O-])cc1 ; CCOC(=O)c1cccc(NC(=O)c2ccccc2)c1]

7-16. (canceled)

17. A method of inhibiting ODC of the medicament molecule according to claim 6, comprising Step 1) construction of ODC prokaryotic expression plasmid, Step 2) ODC protein expression, Step 3) purification of ODC protein, Step 4) detection of ODC protein activity and Step 5) detection of inhibitory activity of inhibitor for ODC protein, wherein,
in Step 1), ODC gene sequence is inserted into pET28a plasmid by BamH I and Xho I cleavage sites to construct pET28a-hODC plasmid, which is verified by DNA sequencing:
in Step 2), the plasmid pET28a-hODC constructed in the step 1) is transformed into Escherichia coli BL21 strain by CaCl2 method and screened by kanamycin, and then strains grown on a kanamycin-containing Luria-Bertani (LB) culture plate are inoculated to kanamycin-containing LB liquid medium, cultured to logarithmic phase at 37° C. and 250 rpm, and then IPTG is added to 0.5 mM for induced expression 4 hours at 28° C., finally, centrifuged to collect bacteria;
in Step 3), the bacteria collected in step 2) are re-suspended with lysate solution, then cells are lysed by an ultrasonic method; after lysis bufferis centrifuged at 12000 rpm/min at 4° C., a supernatant is retained; finally, the supernatant is bound and purified using Ni-NTA His labeled protein binding packing, to get ODC protein; the ODC protein elation buffer is 50 mM Tris/HCl, pH 8.0, 300 mM NaCl, 1 mM DTT, 100 mM imidazole;
in Step 4), 400 μL substrate reaction mixture and 50 ug ODC protein are added to a first EP tube, mixed evenly, and the first EP tube is placed in 37° C. water bath for 30 min; 400 uL 10% PDA is added to terminate the reaction, centrifoged 5 min at 5000 rpm at room temperature, then 100 uL supernatant is fetched and mixed with 200 uL of 4 mol/L NaOH, 400 uL of n-amyl alcohol is added to mix well, centrifuged 5 mm at 2000 rpm, then 200 uL of the supernatant is transferred to a second EP tube, and 200 uL of 0.1 mol/L sodium tetraborate (pH 8.0) is added to mix evenly, and 200 uL of 10 mmol/L trinitrobenzene sulfonic acid is added to mix fully, and then 400 uL DMSO is added to fix folly for 1 min, centrifuged 5 min at 3000 rpm; finally the supernatant is fetched to 96-well plate and its absorbance at 426 nm is detected by a microplate reader, to get an OD value without adding enzyme;
in Step 5), according to procedures in step 4), after adding 400 μL substrate reaction mixture, a small molecule ODC inhibitor is added immediately, and subsequent procedures ate the same as the step 4);
ODC inhibition ratio is calculated according to following formula:
control difference=mean OD value of a control group adding the inhibitor−mean OD value of a control group without adding inhibitor, of which, the inhibitor added in step 5) in the control group is DFMO inhibitor;
experimental difference=mean OD value of an experiment group adding the small molecule inhibitor−mean OD value of an experiment group without adding the small molecule inhibitor,
ODC inhibition ratio=[(control difference−experimental difference)/control difference]×100%.

18. The method according to claim 17, wherein the lysis buffer described in step 3) is a mixture of 50 mM Tris/HCl, pH 8.0, 300 mM NaCl, 1 mM DTT, 1 mM PMSF and 5mM imidazole.

19. The method according to claim 17, wherein the substrate reaction mixture in step 4) is a mixture that dissolves 17.57 ul β-mercaptoethanol 55.84 mg of 1.5 mM EDTA disodium salt, 75 nM PLP stock solution and 2 mM ornithine hydrochloride in 150 mM PBS (pH 7.1).

20. The method of claim 17, wherein the ODC is a human ODC, a non-human ODC or a protein highly homologous to a putrescine substrate and PLP binding site of human ODC.

21. (canceled)


SEQUENCE LISTING (1 sequence)

SEQ ID NO: 1 - DNA, 1386 bp, Homo sapiens
atgaacaactttggtaatgaagagtttgactgccacttcctcgatgaaggttttactgccaaggacattctggaccagaaaattaatgaagtttcttcttctgatgataaggatgccttctatgtggcagacctgggagacattctaaagaaacatctgaggtggttaaaagctctccctcgtgtcacccccttttatgcagtcaaatgtaatgatagcaaagccatcgtgaagacccttgctgctaccgggacaggatttgactgtgctagcaagactgaaatacagttggtgcagagtctgggggtgcctccagagaggattatctatgcaaatccttgtaaacaagtatctcaaattaagtatgctgctaataatggagtccagatgatgacttttgatagtgaagttgagttgatgaaagttgccagagcacatcccaaagcaaagttggttttgcggattgccactgatgattccaaagcagtctgtcgtctcagtgtgaaattcggtgccacgctcagaaccagcaggctccttttggaacgggcgaaagagctaaatatcgatgttgttggtgtcagcttccatgtaggaagcggctgtaccgatcctgagaccttcgtgcaggcaatctctgatgcccgctgtgtttttgacatgggggctgaggttggtttcagcatgtatctgcttgatattggcggtggctttcctggatctgaggatgtgaaacttaaatttgaagagatcaccggcgtaatcaacccagcgttggacaaatactttccgtcagactctggagtgagaatcatagctgagcccggcagatactatgttgcatcagctttcacgcttgcagttaatatcattgccaagaaaattgtattaaaggaacagacgggctctgatgacgaagatgagtcgagtgagcagacctttatgtattatgtgaatgatggcgtctatggatcatttaattgcatactctatgaccacgcacatgtaaagccccttctgcaaaagagacctaaaccagatgagaagtattattcatccagcatatggggaccaacatgtgatggcctcgatcggattgttgagcgctgtgacctgcctgaaatgcatgtgggtgattggatgctctttgaaaacatgggcgcttacactgttgctgctgcctctacgttcaatggcttccagaggccgacgatctactatgtgatgtcagggcctgcgtggcaactcatgcagcaattccagaaccccgacttcccacccgaagtagaggaacaggatgccagcaccctgcctgtgtcttgtgcctgggagagtgggatgaaacgccacagagcagcctgtgcttcggctagtattaatgtgtag